(19) United States
(12) Patent Application Publication
(10) Pub. No.: US 2001/0039297 A1
(43) Pub. Date: Nov. 8, 2001

(54) COMPOSITIONS AND METHOD FOR IMPROVING INSULIN SENSIVITY AND GLUCOSE METABOLISM IN INDIVIDUALS WITH IMPAIRED FASTING GLUCOSE OR IMPAIRED GLUCOSE TOLERANCE

(75) Inventor: GEOFFREY ALLAN, RICHMOND, VA (US)

Correspondence Address: STERNE KESSLER GOLDSTEIN & FOX, 1100 NEW YORK AVE NW, SUITE 600, WASHINGTON, DC, 200053934

(73) Assignee: Insmed Pharmaceuticals, Inc.

(21) Appl. No.: 09/080,459

(22) Filed: May 19, 1998

(51) Int. Cl. A61K 31/715; A61K 31/015

(52) U.S. Cl. 514/763; 514/866

(57) ABSTRACT

The current invention relates to the use of D-chiro-inositol in improving insulin sensitivity and glucose metabolism in individuals with abnormal glucose tolerance who do not have Type 2 diabetes. D-chiro-inositol and related oral compositions may be used in treating abnormal glucose tolerance and metabolism and insulin sensitivity in mammals, and thus help prevent the progression to insulin-resistant Type 2 diabetes.

### FIELD OF THE INVENTION

The current invention relates to the use of D-chiro-inositol in improving insulin sensitivity and glucose metabolism in individuals with abnormal glucose tolerance who do not have Type 2 diabetes. D-chiro-inositol and related oral compositions may be used in treating abnormal glucose tolerance and metabolism and insulin sensitivity in mammals, and thus help prevent the progression to insulin-resistant Type 2 diabetes.

### BACKGROUND OF THE INVENTION

Abnormal glucose tolerance refers to metabolic stages intermediary to normal glucose homeostasis and Type 2 diabetes; this includes conditions like impaired glucose tolerance (IGT) and impaired fasting glucose (IFG) where glucose values are above the conventional normal range and are often accompanied by a decrease in insulin sensitivity. Impaired glucose tolerance (IGT) and impaired fasting glucose (IFG) are transient, intermediate stages in the development of Type 2 diabetes. Within ten years of diagnosis, approximately 30% of IGT subjects will progress to Type 2 diabetes and potentially to health problems that accompany this disease, including retinopathy, nephropathy, and peripheral neuropathy. In addition, abnormal glucose tolerance and decreased insulin sensitivity are associated with a high risk for the development of hypertension, dyslipidemia and an increase incidence of coronary artery disease.

Abnormal glucose tolerance and decreased insulin sensitivity can be attributed to a wide range of causes including obesity, age, physical activity level, certain medication or drugs, genetic factors, and some endocrine related disorders. The truncal distribution of weight as determined by a high waist to hip ratio (WHR) is a good predictor of abnormal insulin sensitivity, and there is an excellent correlation between a high body mass index (BMI) and decreased insulin sensitivity. Approximately 33% of the population in the United States is obese and the majority of these individuals have decreased insulin sensitivity, are hyperinsulinemic, and often have abnormal glucose tolerance.

Impaired fasting glucose (IFG) is defined by the American Diabetes Association as a fasting blood glucose in the range of 110 mg/dL to 125 mg/dL. IFG is determined by analysis of a plasma sample for glucose after a 10-16 hour fast. This is an easy and quick way to determine if there is a problem with glucose tolerance and metabolism.

Impaired glucose tolerance, as defined by the World Health Organization, is determined by the administration of a standard oral glucose tolerance test (OGTT) (World Health Org., Diabetes Mellitus, Tech. Rep. Ser., no. 727 (1985)). During an OGTT, a measured amount of glucose (75 grams) is given to a fasted individual and blood glucose levels are measured every 30 minutes, usually for 2 or 3 hours. In a individual with normal glucose tolerance, the blood glucose values will rise during the first part of the test and then rapidly return to basal levels. In an IGT individual the post prandial glucose levels will rise above the normal range, producing a 2-hour glucose value of 140-199 mg/dL, and return to basal levels at a slow rate.

Abnormal glucose tolerance is caused in part by inadequate utilization of glucose in the periphery—at the site of the muscles. In addition, high fasting glucose values, seen with impaired fasting glucose, suggest that hepatic glucose production is not being effectively regulated. The underlying cause of this abnormal glucose tolerance is characterized by a decrease in insulin sensitivity.

Insulin sensitivity is a measurement of insulin's ability to produce a biological response; specifically, in the case of glucose regulation, insulin sensitivity is a measurement of insulin's ability to promote the clearance and utilization of glucose. A decrease in insulin sensitivity will result in a prolonged elevation of glucose levels and the release of additional insulin to try and achieve a euglycemic state. This compensatory hyperinsulinemia will effect insulin's ability to suppress lipolysis in adipose tissue, thus causing an increase in free fatty acids and ultimately resulting in the disruption of normal lipid profiles which could lead to coronary artery disease. The increase in free fatty acids will also inhibit insulin-stimulated glucose utilization in the muscle and stimulate hepatic gluconeogenesis. This leads to increased blood glucose and will eventually result in the development of impaired glucose tolerance or impaired fasting glucose and ultimately, if unchecked, the development of Type 2 diabetes. Improving insulin sensitivity will restore overall glucose control and decrease the risk of cardiovascular disease.

The level of insulin sensitivity can be measured to varying degrees by three methods: fasting plasma insulin, the euglycemic clamp, and the frequently sampled intravenous glucose tolerance test (FSIGTT). With the use of any of these techniques there is a wide range of insulin sensitivity, with overlapping values characterizing individuals with normal, abnormal glucose tolerance and Type 2 diabetes.

One method of determining insulin sensitivity is by fasting plasma insulin values. Simply, a high fasting insulin value correlates with decreased insulin sensitivity.

The euglycemic clamp test procedure involves infusing glucose at a variable rate in order to obtain a constant plasma glucose concentration. This rate of glucose infusion is equal to the overall rate of basal glucose disposal in the body. When insulin is also infused, the glucose infusion rate reflects insulin mediated glucose uptake. This is a precise and reproducible way to determine insulin sensitivity, albeit the euglycemic clamp method is time-consuming and complicated to perform.

The third method of determining insulin sensitivity involves frequently collecting blood samples for glucose and insulin during an intravenous glucose tolerance test, and analyzing the glucose and insulin dynamics using a minimal mathematical model developed by Bergman (Bergman et. al., J. Clin. Invest. 68:1456-1467 (1981)). This test can be modified by the injection of tolbutamide or exogenous insulin to boost the insulin response and improve the correlation with the euglycemic clamp. The FSIGTT provides an accessible way to determine the insulin sensitivity index (S₁). The more insulin insensitive a subject is, the lower the calculated S₁. As insulin sensitivity is improved, the S₁ value is increased—thus a higher S₁ value corresponds to greater insulin sensitivity.

Improving insulin sensitivity and glucose tolerance will help delay and even prevent the onset of Type 2 diabetes mellitus, and thus improve the quality of life by preventing acute and long-term complications, reducing mortality and treating accompanying disorders of those at risk for Type 2 diabetes. This invention involves, in one embodiment, the administration of certain isomers of inositol, namely D-chiro-inositol and derivatives and metabolites thereof, that will improve insulin sensitivity and glucose tolerance, and thereby help prevent or delay the onset of Type 2 diabetes and all of its tremendous medical, financial and socioeconomic ramifications.

### SUMMARY OF THE INVENTION

Certain isomers of inositol, namely D-chiro-inositol and derivatives and metabolites thereof and compounds containing D-chiro-inositol or a derivative or metabolite thereof, have significant effects on mammalian endocrinology and metabolism. More specifically, D-chiro-inositol and derivatives and metabolites thereof and compounds containing D-chiro-inositol or a derivative or metabolite thereof, significantly improve glucose metabolism and disposal, and increase insulin sensitivity when administered to subjects with reduced insulin sensitivity and abnormal glucose tolerance, who do not have Type 2 diabetes.

Accordingly, the first embodiment of the present invention is directed to a composition, which comprises an effective amount of D-chiro-inositol, or a suitable derivative or metabolite thereof, or a compound containing D-chiro-inositol or a derivative or metabolite thereof, and an acceptable carrier, for improving insulin sensitivity and glucose metabolism in mammals suffering from impaired glucose tolerance or impaired fasting glucose.

A second embodiment of the present invention is directed to a method for improving insulin sensitivity and restoring normal glucose tolerance and metabolism in individuals with impaired glucose tolerance or impaired fasting glucose, which comprises the step of administering to a mammal an effective amount of D-chiro-inositol, or a suitable derivative or metabolite thereof or a compound containing D-chiro-inositol or a derivative or metabolite thereof or a pharmaceutical composition containing D-chiro-inositol or a derivative or metabolite thereof.

A third embodiment of the present invention is directed to a method for treating mammalian metabolic diseases characterized by abnormal glucose tolerance and decreased insulin sensitivity, such as impaired glucose tolerance and impaired fasting glucose, which comprises the step of administering to a mammal in need thereof an effective amount of D-chiro-inositol, or a suitable derivative or metabolite thereof or a compound containing D-chiro-inositol or a derivative or metabolite thereof.

It is to be understood that both the foregoing general description and the following detailed description are exemplary and explanatory only and are intended to provide further explanation of the invention as claimed.

### BRIEF DESCRIPTION OF THE FIGURES

FIG. 1 is a graph showing an increase in insulin sensitivity (S₁) calculated by the Bergman minimal mathematical model, in 6 out of 9 obese male subjects after a single dose (300-1200 mg) of D-chiro-inositol.

FIG. 2 is a graph showing statistically significant improvement in glucose tolerance in 7 subjects with abnormal glucose tolerance in response to an oral glucose tolerance test after 7-14 days administration of 1200 mg/day D-chiro-inositol. (Data is means ±SE and the * represents significance at p<0.05).

FIG. 3 is a graph showing that D-chiro-inositol administration (1200 mg) for 7-14 days does not alter the glucose profile during an oral glucose tolerance test in individuals with normal glucose tolerance (n=11).

### DETAILED DESCRIPTION OF THE PREFERRED EMBODIMENTS

In the first preferred embodiment, the present invention is directed to compositions and methods for improving insulin sensitivity and glucose metabolism in mammals with impaired glucose regulation, specifically impaired glucose tolerance and impaired fasting glucose.

The inventive composition comprises an effective amount of D-chiro-inositol, or a suitable derivative or metabolite thereof, or a compound containing D-chiro-inositol or a derivative or metabolite thereof, and an acceptable carrier. Preferably, the inventive composition comprises an effective amount of D-chiro-inositol or a compound containing D-chiro-inositol.

The inventive method comprises the step of administering to a mammal in need thereof an effective amount of D-chiro-inositol, or a suitable derivative or metabolite thereof, or a compound containing D-chiro-inositol or a derivative or metabolite thereof. Preferably, the inventive method comprises the step of administering to a mammal in need thereof an effective amount of D-chiro-inositol or a compound containing D-chiro-inositol.

While the inventive composition preferably comprises D-chiro-inositol or a compound containing D-chiro-inositol, suitable derivatives and/or metabolites of D-chiro-inositol, or compounds containing derivatives or metabolites of D-chiro-inositol, may also be employed.

As used herein, a “suitable derivative or metabolite” of D-chiro-inositol is a compound based on or derived from the D-chiro-inositol moiety. Illustrative examples of suitable derivatives and metabolites of D-chiro-inositol include, but are not limited to, the following: D-chiro-inositol phosphates; D-chiro-inositol esters, preferably acetates; D-chiro-inositol ethers, preferably lower alkyl ethers; D-chiro-inositol acetals; and D-chiro-inositol ketals.

As used herein, a “compound containing D-chiro-inositol” is any compound that contains the D-chiro-inositol moiety. Illustrative examples of D-chiro-inositol containing compounds include, but are not limited to, the following: polysaccharides containing D-chiro-inositol and one or more additional sugars, such as glucose, galactose and mannose, or derivatives thereof, such as glucosamine, galactosamine and mannitol; D-chiro-inositol phospholipids; and complexes or chelates of D-chiro-inositol with one or more metal ions and the like.

The active agent in the inventive composition (i.e. D-chiro-inositol or a suitable derivative or metabolite thereof or a compound containing D-chiro-inositol, or a derivative or metabolite thereof) may be used alone or in admixture with one or more additional active agents. For example, a composition according to the first embodiment of the present invention may include D-chiro-inositol and a compound containing D-chiro-inositol in admixture.

As used herein, an “acceptable carrier” is a non-toxic solid, semisolid or liquid filler, diluent, encapsulating material or formulation auxiliary of any type known to those skilled in the art for use in pharmaceuticals.

When administered to a mammal, the inventive compositions may be administered orally, rectally, parenterally, intracistemally, intravaginally, intraperitoneally, topically (as by powders, ointments, drops or transdermal patch), bucally, or as an oral or nasal spray. Preferably, the inventive compositions are administered orally, for example in the form of a tablet or capsule.

As used herein, the term “parenteral” refers to modes of administration which include intravenous, intramuscular, intraperitoneal, intrastemal, subcutaneous and intraarticular injection and infusion.

The compositions of the present invention are also suitably administered by sustained-release systems. Suitable examples of sustained-release compositions include semi-permeable polymer matrices in the form of shaped articles, e.g., films, or mirocapsules. Sustained-release matrices include polylactides (U.S. Pat. No. 3,773,919, EP 58,481), copolymers of L-glutamic acid and gamma-ethyl-L-glutamate (Sidman et al., Biopolymers 22:547-556 (1983)), poly (2-hydroxyethyl methacrylate) (Langer et al., J. Biomed. Mater. Res. 15:167-277 (1981), and Langer, Chem. Tech. 12:98-105 (1982)), ethylene vinyl acetate (Langer et al.) or poly-D-(-)-3-hydroxybutyric acid (EP 133,988).

Sustained-release compositions also include liposomally entrapped compounds. Liposomes containing one or more of the compounds of the present invention may be prepared by methods known per se: DE 3,218,121; Epstein et al., Proc. Natl. Acad. Sci. (USA) 82:3688-3692 (1985); Hwang et al., Proc. Natl. Acad. Sci. (USA) 77:4030-4034 (1980); EP 52,322; EP 36,676; EP 88,046; EP 143,949; EP 142,641; Japanese Pat. Appl. 83-118008; U.S. Pat. Nos. 4,485,045 and 4,544,545; and EP 102,324. Ordinarily, the liposomes are of the small (about 200-800 Angstroms) unilamellar type in which the lipid content is greater than about 30 mol. percent cholesterol, the selected proportion being adjusted for the optimal therapy.

For parenteral administration, in one embodiment, the composition of the present invention is formulated generally by mixing an effective amount of the active agent at the desired degree of purity, in a unit dosage injectable form (solution, suspension, or emulsion), with an acceptable carrier, i.e., one that is non-toxic to recipients at the dosages and concentrations employed and is compatible with other ingredients of the formulation. For example, the formulation preferably does not include strong oxidizing agents and other compounds that are known to be deleterious to the active agent.

Generally, the formulations are prepared by contacting the active agent uniformly and intimately with liquid carriers or finely divided solid carriers or both. Then, if necessary, the product is shaped into the desired formulation. When the carrier is a parenteral carrier, it is preferably a solution that is isotonic with the blood of the recipient. Examples of such carrier vehicles include water, saline, Ringer's solution, and dextrose solution. Non-aqueous vehicles such as fixed oils and ethyl oleate are also useful herein, as well as liposomes.

The carrier suitably contains minor amounts of additives such as substances that enhance isotonicity and chemical stability. Such materials are non-toxic to recipients at the dosages and concentrations employed, and include buffers such as phosphate, citrate, succinate, acetic acid, and other organic acids or their salts; antioxidants such as ascorbic acid; low molecular weight (less than about ten residues) polypeptides, e.g., polyarginine or tripeptides; proteins, such as serum albumin, gelatin, or immunoglobulins; hydrophilic polymers such as polyvinylpyrrolidone; amino acids, such as glycine, glutamic acid, aspartic acid, or arginine; monosaccharides, disaccharides, and other carbohydrates including cellulose or its derivatives, glucose, mannose, or dextrins; chelating agents such as EDTA; sugar alcohols such as mannitol or sorbitol; counterions such as sodium; and/or nonionic surfactants such as polysorbates, poloxamers, or PEG.

The compositions of the present invention are typically formulated in such vehicles at a concentration of active agent of about 1 mg/mL to 240 mg/mL, preferably 30 to 120 mg/mL.

It will be understood that the use of certain of the foregoing excipients, carriers, or stabilizers may result in the formation of salts depending upon the particular substitutent(s) on the active agent.

The compositions of the present invention ordinarily will be stored in unit or multi-dose containers, for example, sealed ampules or vials, as an aqueous solution or as a lyophilized formulation for reconstitution. As an example of a lyophilized formulation, 10-mL vials are filled with 5 mL of sterile-filtered 1% (w/v) aqueous solution, and the resulting mixture is lyophilized. The infusion solution is prepared by reconstituting the lyophilized composition using bacteriostatic Water-for-Injection.

The compositions of the present invention will be formulated and dosed in a fashion consistent with good medical practice, taking into account the clinical condition of the individual patient (especially the side effects of treatment with the active agent), the site of delivery of the composition, the method of administration, the scheduling of administration, and other factors known to practitioners. The “effective amount” of active agent for the purposes of the present invention is determined in view of such considerations. Those skilled in the art can readily determine empirically an appropriate “effective amount” for a particular patient.

The key factor in selecting an appropriate dose is the result obtained, as measured, for example, by increases or decreases in blood glucose levels, insulin levels and sensitivity in the patient. The length of treatment needed to observe changes and the interval following treatment for responses to occur appears to vary depending on the desired effect.

As a general proposition, the total effective amount of active agent administered per dose will be in the range of about 0.5 mg/kg/day to 1,000 mg/kg/day of mammalian patient body weight, although, as noted above, this will be subject to therapeutic discretion. More preferably, this dose is at least 0.5 mg/kg/day, and most preferably for humans between about 1 and 50 mg/kg/day.

In a most preferred embodiment, the inventive compositions are formulated for oral delivery according to the methods known to those skilled in the art. For example, the active agent is combined with suitable sweetening agents, flavoring agents, coloring agents and preserving agents, in order to obtain a palatable preparation. Tablets, capsules, powders, granules, and the like for oral administration may contain the active agent in admixture with acceptable additives or excipients. Such forms may be prepared by mixing the active agent(s) with one or more additives and excipients, such as inert diluents, granulating agents, disintegrating agents, binding agents and/or lubricating agents, under suitable conditions.

Acceptable additives and excipients are known to those skilled in the art (see, e.g., Remington's Pharmaceutical Sciences, 18th ed., A. Gennaro, ed., Mack Publishing Company, Easton, Pa. (1990)). Illustrative examples of acceptable additives and excipients for oral compositions include, but are not limited to, water, non-fat dry milk, maltodextrin, sugar, corn syrup, sodium caseinate, soy protein isolate, calcium caseinate, potassium citrate, sodium citrate, tricalcium phosphate, magnesium chloride, sodium chloride, lecithin, potassium chloride, choline chloride, ascorbic acid, potassium hydroxide such as calcium carbonate, sodium carbonate, lactose, calcium phosphate, carrageenan, vitamin E, zinc sulfate, ferrous sulfate, niacinamide, biotin, vitamin A, calcium pantothenate, copper gluconate, magnesium sulfate, vitamin K, potassium iodide, folic acid, vitamin D, vanillin, cocoa, polysorbate 80, polysorbate 60, magnesium oxide, riboflavin, pyridoxine hydrochloride, cyanocobalamin, aspartame, thiamine, cellulose, methyl cellulose, hydroxypropyl methylcellulose, alginate, polyoxyelthylene sorbitol monooleate, polyoxyethylene stearate, gum acacia, gum tagacanth, polyvinylpyrrolidone, gelatin, calcium carbonate, calcium phosphate, kaolin, starch, and the like.

When administered orally, the inventive composition preferably contains from about 1 mg to about 1200 mg of active ingredient. In the case of D-chiro-inositol, the inventive composition preferably contains from about 10 mg to about 900 mg of DCI, more preferably about 30 mg to about 600 mg and most preferably about 100 mg to about 300 mg.

In the second preferred embodiment, the present invention is directed to compositions and methods for improving insulin sensitivity and glucose metabolism in mammals with impaired glucose tolerance and impaired fasting glucose.

In this embodiment, the inventive composition comprises an effective amount of D-chiro-inositol, or a suitable derivative or metabolite thereof, or a compound containing D-chiro-inositol or a derivative or metabolite thereof, and an acceptable carrier. Preferably, the inventive composition comprises D-chiro-inositol.

The inventive method comprises the step of administering to a mammal in need thereof an effective amount of D-chiro-inositol, or a suitable derivative or metabolite thereof, or a compound containing D-chiro-inositol or a derivative or metabolite thereof. Preferably, the inventive method comprises the step of administering to a mammal an effective amount of D-chiro-inositol.

The following examples are illustrative only and are not intended to limit the scope of the invention as defined by the appended claims. It will be apparent to those skilled in the art that various modifications and variations can be made in the methods of the present invention without departing from the spirit and scope of the invention. Thus, it is intended that the present invention cover the modifications and variations of this invention provided they come within the scope of the appended claims and their equivalents.

All patents and publications referred to herein are expressly incorporated by reference.

### EXAMPLE 1

A clinical study of D-chiro-inositol was performed in obese men. This was a three phase, double blind, single oral dose crossover study with subjects receiving either D-chiro-inositol (300-1200 mg) or placebo in a randomized crossover fashion. Treatments were administered to subjects in a fasted state 2 hours before the tolbutamide-modified frequently sampled intravenous glucose tolerance test (FSIGTT) began.

The insulin sensitivity index (S₁) was derived using the minimal model analysis of plasma glucose and insulin concentrations sampled during a full tolbutamide-modified FSIGTT. The data is summarized below:

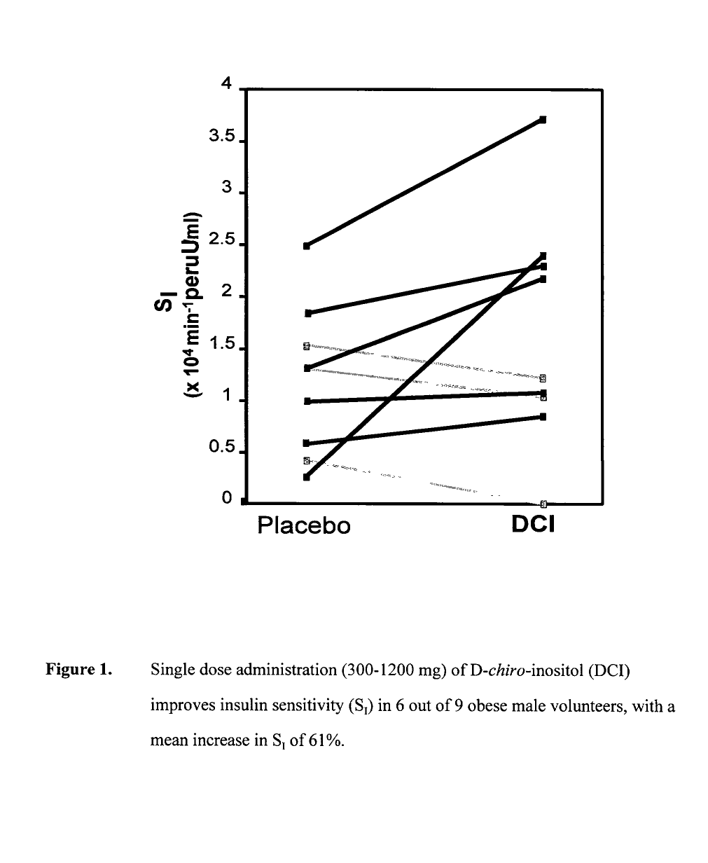

6 out of 9 subjects demonstrated an increase in insulin sensitivity following D-chiro-inositol treatment (FIG. 1)

The mean increase in insulin sensitivity was 61% after D-chiro-inositol treatment.

### EXAMPLE 2

A clinical study utilizing D-chiro-inositol was performed in obese men and women with normal and abnormal glucose tolerance. Eighteen volunteers received D-chiro-inositol (1200 mg/day) for 7 to 14 days. An oral glucose tolerance test was performed at the beginning of the study, prior to treatment, and at the end of the study, after D-chiro-inositol treatment. Results of the study are summarized below:

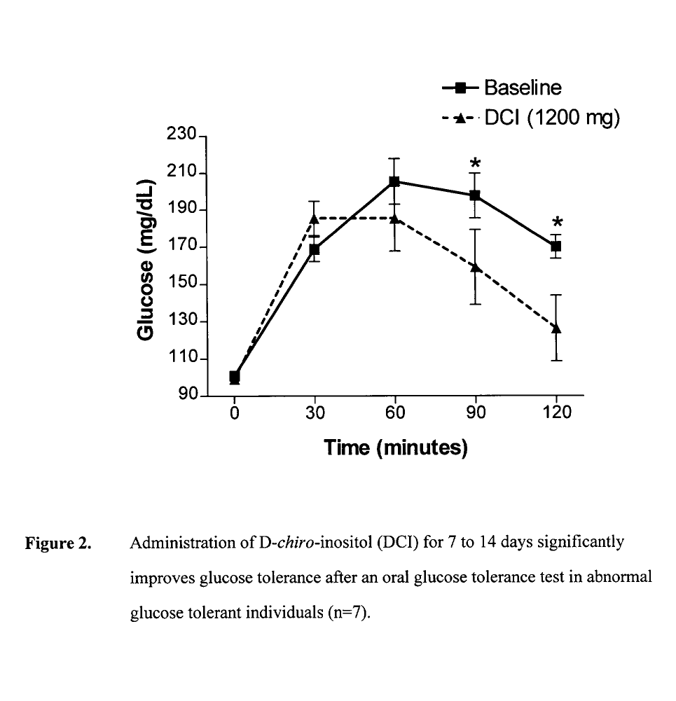

Seven subjects had an abnormal, elevated glucose response to the glucose load. This glucose response was significantly improved after D-chiro-inositol treatment (FIG. 2).

D-chiro-inositol treatment produced a statistically significant reduction in the 90 and the 120 minute glucose values in these abnormal glucose tolerant subjects.

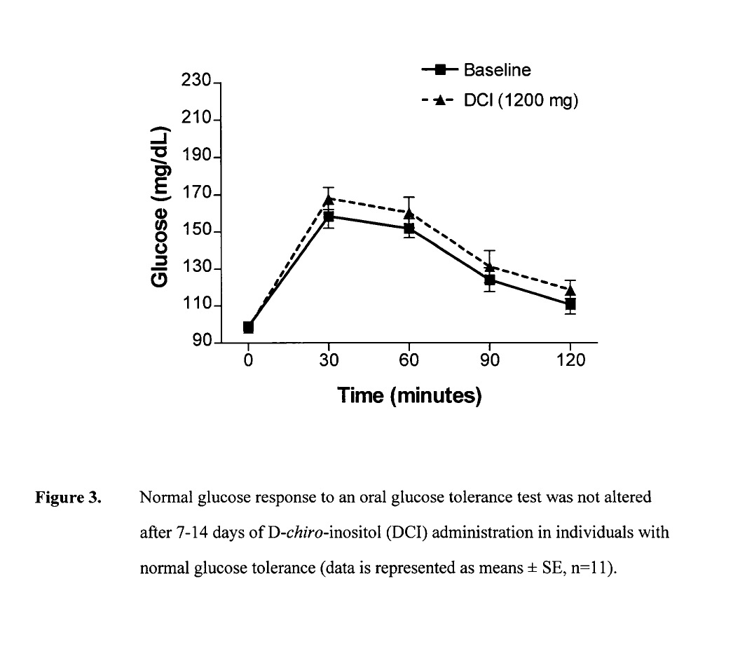

In contrast to the subjects above, eleven volunteers had a normal glucose response to the glucose load. In these subjects the glucose values were not modified as a result of D-chiro-inositol treatment (FIG. 3).

What is claimed is:

1. A method for improving insulin sensitivity and glucose metabolism in mammals afflicted with abnormal glucose tolerance, said method comprising the step of administering to a mammal an effective amount of D-chiro-inositol or a derivative or metabolite of D-chiro-inositol, or a compound containing D-chiro-inositol or a derivative or metabolite thereof.

2. The method according to claim 1, wherein said derivative or metabolite of D-chiro-inositol is selected from the group consisting of D-chiro-inositol phosphates, D-chiro-inositol esters, D-chiro-inositol ethers, D-chiro-inositol acetals, and D-chiro-inositol ketals.

3. The method according to claim 1, wherein D-chiro-inositol is administered.

4. The method according to claim 1, wherein said compound containing D-chiro-inositol is selected from the group consisting of polysaccharides containing D-chiro-inositol, D-chiro-inositol phospholipids and complexes or chelates of D-chiro-inositol with one or more metal ions.

5. A method for treating abnormal glucose tolerance and for improving insulin sensitivity in individuals with impaired glucose tolerance or impaired fasting glucose, which comprises the step of administering to a mammal in need thereof an effective amount of D-chiro-inositol or a derivative or metabolite of D-chiro-inositol or a compound containing D-chiro-inositol

6. The method according to claim 5, wherein said derivative or metabolite of D-chiro-inositol is selected from the group consisting of D-chiro-inositol phosphates, D-chiro-inositol esters, D-chiro-inositol ethers, D-chiro-inositol acetals, and D-chiro-inositol ketals.

7. The method according to claim 5, wherein D-chiro-inositol is administered.

8. A pharmaceutical composition for treating abnormal glucose tolerance and for improving insulin sensitivity comprising an effective amount of D-chiro-inositol, or a derivative or metabolite of D-chiro-inositol, or a compound containing D-chiro-inositol or a derivative or metabolite thereof, and an acceptable carrier.

9. The pharmaceutical composition of claim 8, wherein said composition comprises D-chiro-inositol.

10. A pharmaceutical composition for improving insulin sensitivity and glucose metabolism in individuals with impaired glucose tolerance and impaired fasting glucose, said composition comprising an effective amount of D-chiro-inositol, or a derivative or metabolite of D-chiro-inositol, or a compound containing D-chiro-inositol or a derivative or metabolite thereof, and an acceptable carrier.

11. The pharmaceutical composition of claim 10, wherein said composition comprises D-chiro-inositol.
